(19) United States
(12) Patent Application Publication
(10) Pub. No.: US 2007/0258930 A1
(43) Pub. Date: Nov. 8, 2007

(54) Water-in-oil emulsion for the treatment of keratin fibers, comprising a cyanoacrylate monomer

(76) Inventor: Gabin Vic, Venette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,922

(22) Filed: Apr. 13, 2007

Related U.S. Application Data
(60) Provisional application No. 60/796,216, filed on May 1, 2006.

(30) Foreign Application Priority Data
Apr. 13, 2006 (FR) 0603328

(51) Int. Cl. A61K 8/81

(52) U.S. Cl. 424/70.7; 424/70.16

(57) ABSTRACT

The present disclosure relates to a water-in-oil emulsion comprising an aqueous phase, at least one surfactant and at least one monomer polymerization inhibitor and an oily phase comprising at least one cyanoacrylate monomer. The water-in-oil emulsion of the present disclosure facilitates the application of cyanoacrylate monomers to keratin fibers.

This application claims benefit of U.S. Provisional Application No. 60/796,216, filed May 1, 2006, the contents of which are incorporated herein by reference. This application also claims benefit of priority under 35 U.S.C. § 119 to French Patent Application No. FR 06/03328, filed Apr. 13, 2006, the contents of which are also incorporated herein by reference.

The present disclosure relates to a water-in-oil emulsion for the treatment of keratin fibers, for example keratin fibers such as the hair, comprising at least one cyanoacrylate monomer.

Hair treatment compositions based on compositions comprising electrophilic monomers are described in French patent application FR 2 833 489. Such a composition allows perfectly coated hair to be obtained.

Because water is a polymerization initiator, use of these electrophilic monomers, including for example, cyanoacrylates, requires the use of an anhydrous formulation. This constraint limits the formulation of water-soluble additives in compositions containing these monomers. For example, when water-soluble additives are necessary, they may be applied as a pretreatment before the application of the electrophilic monomer. As a result, this makes the treatment of the keratin fibers more complex.

The present disclosure relates to new compositions for treating keratin fibers which make it possible to obtain permanent coatings on keratin fibers using cosmetic additives, without it being necessary to pretreat the fibers, while at the same time conserving the quality of the coating and the properties of the additives introduced.

The present disclosure thus relates to a water-in-oil emulsion comprising an aqueous phase, at least one surfactant, at least one monomer polymerization inhibitor and an oily phase comprising at least one cyanoacrylate monomer capable of polymerizing anionically in the presence of a nucleophilic agent.

The water-in-oil emulsion of the present disclosure makes it possible to conserve the cyanoacrylate monomer under conditions such that it is no longer necessary to carry out a pretreatment of keratin fibers with an aqueous composition before the application of the cyanoacrylate. As a result, the application of the monomer is facilitated, and the time necessary to treat the fibers is reduced.

The present disclosure also relates to a process for treating keratin fibers, comprising applying the emulsion of the present disclosure to keratin fibers, as well as the use of this emulsion for the treatment of keratin fibers, such as the hair.

The emulsion of the present disclosure comprises an oily phase. This oily phase comprises at least one cyanoacrylate monomer. It may also comprise at least one organic solvent. In at least one embodiment, the organic solvents are chosen from compounds that are liquid at a temperature of 25° C. and pressure of less than 10⁵ Pa (760 mm Hg).

In at least one embodiment, the oily phase comprises a mixture of at least one organic solvent and at least one cyanoacrylate monomer.

For the purpose of the present disclosure, the at least one cyanoacrylate monomer and at least one organic solvent are different from each other.

The at least one organic solvent may, for example, be chosen from:
aromatic alcohols, for example benzyl alcohol;
liquid fatty alcohols, for example C₁₀-C₃₀ fatty alcohols;
modified or unmodified polyols, for example glycerol, glycol, propylene glycol, dipropylene glycol, butylene glycol and butyl diglycol;
volatile silicones, for example cyclopentasiloxane, cyclohexasiloxane, polydimethylsiloxanes that are optionally modified with at least one alkyl, amine, imine, fluoroalkyl, carboxylic, betaine, and/or quaternary ammonium function;
liquid modified polydimethylsiloxanes;
mineral, organic and plant oils;
alkanes, for example C₅ to C₁₀ alkanes;
liquid fatty acids;
liquid fatty esters, for example liquid fatty alcohol benzoates or salicylates.

In at least one embodiment, the organic solvent is chosen from organic oils; silicones, including but not limited to volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil; and organic compounds, including, but not limited to C₅-C₁₀ alkanes, acetone, methyl ethyl ketone, esters of liquid C₁-C₂₀ acids and C₁-C₈ alcohols,such as methyl acetate, butyl acetate, ethyl acetate and isopropyl myristate, dimethoxyethane, diethoxyethane, liquid C₁₀-C₃₀ fatty alcohols such as oleyl alcohol, esters of liquid C₁₀-C₃₀ fatty alcohols such as C₁₀-C₃₀ fatty alcohol benzoates, and mixtures thereof; polybutene oil, isononyl isononanoate, isostearyl malate, pentaerythrityl tetraisostearate, tridecyl trimellitate, and the mixture of cyclopentasiloxane (14.7% by weight)/polydimethylsiloxane dihydroxylated in the α and ω positions (85.3% by weight), and mixtures thereof.

In at least one embodiment, the organic solvent comprises a silicone or a mixture of silicones, for example liquid polydimethylsiloxanes and modified liquid polydimethylsiloxanes having a viscosity at 25° C. ranging from 0.1 cst to 1 000 000 cst, for example from 1 cst to 30 000 cst.

Non-limiting mention is made of the following oils and mixtures of oils:

the mixture of α,ω-dihydroxylated polydimethylsiloxane/cyclopentadimethylsiloxane (14.7/85.3) sold by Dow Corning under the name DC 1501 Fluid;

the mixture of α,ω-dihydroxylated polydimethylsiloxane/polydimethylsiloxane sold by Dow Corning under the name DC 1503 Fluid;

the mixture of dimethicone/cyclopentadimethylsiloxane sold by Dow Corning under the name DC 1411 Fluid, or that sold by Bayer under the name SF1214;

the cyclopentadimethylsiloxane sold by Dow Corning under the name DC245 Fluid; and mixtures thereof.

In at least one embodiment of the present disclosure, the oily phase comprises at least one solvent, e.g., a volatile silicone, alone or mixed with another silicone.

In at least one embodiment of the present disclosure, the at least one volatile silicone is chosen from linear or cyclic silicones having a viscosity at ambient temperature and under atmospheric pressure of less than 8 mm²/s (8 cSt).

The viscosity may be measured by capillary viscometry, for example with a capillary viscometer such as an Ubbelohde type capillary viscometer, at a temperature of 25° C., according to ASTM standard D445-97. Viscosity may also be measured, for example, by the “falling-ball” method.

In at least one embodiment, the volatile silicones have a boiling point ranging from 60° C. to 260° C., and may, for example, be chosen from:

(i) cyclic volatile silicones containing from 3 to 7, for example 4 to 5, silicon atoms. As examples of such cyclic volatile silicones, non-limiting mention may be made of octamethylcyclotetrasiloxane sold in under the name “Volatile Silicone 7207” by Union Carbide or “Silbione® 70045 V 2” by Rhodia, decamethylcyclopentasiloxane, commonly called D5, sold under the name “Volatile Silicone 7158” by Union Carbide, “Silbione® 70045 V 5” by Rhodia, or under the name DC245 Fluid by Dow Corning, and mixtures thereof.

Non-limiting mention may also be made of cyclocopolymers of the dimethylsiloxane/methylalkylsiloxane type, such as “Silicone Volatile FZ 3109” sold by the company Union Carbide, having the chemical structure:

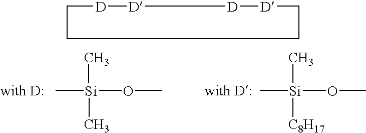

Non-limiting mention is also made of mixtures of cyclic silicones with organosilicon compounds, including but not limited to the mixture of octamethylcyclotetrasiloxane and tetratrimethylsilylpentaerythritol (50/50) and the mixture of octamethylcyclotetrasiloxane and oxy-1,1′-bis-[2,2,2′,2′,3,3′-hexakis(trimethylsilyloxy)]neopentane;

(ii) linear volatile silicones containing 2 to 9 silicon atoms and having a viscosity of less than or equal to 5 mm²/s at 25° C. Non-limiting examples of these linear volatile silicones include decamethyltetrasiloxane sold under the name “SH 200” by the company Toray Silicone and the silicones described in the article published in Cosmetics and Toiletries, Vol. 91, January 76, p. 27-32 - Todd & Byers “Volatile Silicone Fluids for Cosmetics”.

The oily phase may be present in the emulsion in an amount ranging from 5% to 95% by weight, for example, from 10-80% by weight, relative to the total weight of the emulsion. When the oily phase comprises one or more volatile silicones, these silicones may be present in an amount ranging from 0.1% to 30%, for example from 5% to 20% by weight, i.e., from 8% to 15% by weight, relative to the total weight of emulsion.

The oily phase of the composition may further comprise a gelling agent/structuring agent. Non-limiting examples of such gelling/structuring agents include the PSPA silicone polyamides (DP100 and DP15) sold by Dow Chemical, the organic polylauryldimethylsiloxane KSG products and the silicone-containing KSG products sold by Shin-Etsu, dextrin palmitate and inulin stearate known as the RHEOPEARL range from Chiba Flour Milling); the alkyl-chain acrylates sold by Landec, the ethylene octene copolymers sold by Dupont de Nemours, the dibutyl lauryl glutamide sold by Ajinomoto, the disorbene sold by Roquette, the styrene/acrylate copolymers VERSAGEL M5960 and 5670 sold by Penreco, the diblock and triblock kratons sold by Kraton Polymers, the hydroxystearic acid, the jojoba waxes, the fumed silicas sold by Degussa, the silicone waxes sold by Wacker, the polyamide UNICLEAR sold by Arizona Chemical, and bentone.

The surfactant that may be used in the emulsion of the present disclosure may be any surfactant known in the art to form a water-in-oil emulsion. The surfactant may be anionic, amphoteric, cationic or non-ionic.

Non-limiting examples of surfactants that may be used in the compositions of the present disclosure include, but are not limited to non-silicone anionic surfactants, including the salts of the following compounds: alkyl sulphates, alkyl ether sulphates, alkyl-amido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinimates, alkyl sulphoacetates; alkyl ether phosphates; acyl sarcosinates; acyl isethionates and N-acyltaurates. In at least one embodiment, the alkyl or acyl radical of all these various compounds contains from 12 to 20 carbon atoms, and the aryl radical is a phenyl or benzyl group. The salts of these compounds include, but are not limited to alkali metal salts, for example sodium salts, ammonium salts, amine salts, amino alcohol salts and magnesium salts. Among the anionic surfactants, non-limiting mention may be made of the salts of fatty acids including, for example, the salts of oleic acid, ricinoleic acid, palmitic acid or stearic acid, coconut oil acid or hydrogenated coconut oil acid; and acyl lactylates wherein the acyl radical contains 8 to 20 carbon atoms. Weakly anionic surfactants may also be used, including, for example, alkyl-D-galactosiduronic acids and their salts, polyoxyalkylenated ether carboxylic acids and their salts, including those comprising from 2 to 50 ethylene oxide groups, and mixtures thereof. In at least one embodiment, the anionic surfactants of the polyoxyalkylenated ether carboxylic salt or acid type correspond to the following formula (1):
R₁—(OC₂H₄)ₙ—OCH₂COOA (1)
wherein:

R1 is chosen from an alkyl group and an alkylaryl group, and n is an integer or decimal number (average value) ranging from 2 to 24, for example, from 3 to 10. In at least one embodiment, the alkyl radical contains from 6 to 20 carbon atoms, and the aryl group is phenyl.

A is chosen from a hydrogen atom, an ammonium, a sodium, potassium, lithium or magnesium ion or a monoethanolamine or triethanolamine residue. Use may also be made of mixtures of compounds of formula (1), for example, mixtures in which the R1 groups are different from each other.

In at least one embodiment, the anionic surfactant is chosen from alkyl sulphate salts, alkyl ether sulphate salts, and mixtures thereof.

As non-limiting examples of non-silicone amphoteric surfactants that may be used according to the present disclosure, mention may be made of secondary or tertiary aliphatic amine derivatives, wherein the aliphatic radical is a linear or branched chain containing from 8 to 22 carbon atoms and comprises at least one water-soluble anionic group, for example carboxylate, sulphonate, sulphate, phosphate or phosphonate groups; non-limiting mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

As non-limiting examples of amine derivatives that may be used in accordance with the present disclosure, mention may be made of the products sold under the name MIRANOL, as described in U.S. Pat. Nos. 2,528,378 and 2,781,354 and having the structures:
R2-CONHCH2CH2-N(R3)(R4)(CH2COO—) (2)
wherein:

R2 is chosen from alkyl radicals derived from an acid R2-COOH present in hydrolyzed coconut oil, a heptyl radical, a nonyl radical or an undecyl radical,

R3 is a β-hydroxyethyl group and

R4 is a carboxymethyl group; and
R5-CONHCH2CH2-N(B)(C) (3)
wherein:

B is —CH₂CH₂OX′,

C is —(CH₂)_{z}—Y′, with z=1 or 2,

X′ is chosen from the group —CH₂CH₂—COOH or a hydrogen atom,

Y′ is chosen from —COOH or the radical —CH₂—CHOH—SO₃H,

R5 is an alkyl radical of an acid R5-COOH present in hydrolyzed linseed oil or coconut oil, an alkyl radical, such as a C₇, C₉, C₁₁ or C₁₃ alkyl radical, a C₁₇ alkyl radical and its iso form, or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, Cocoamphodipropionic acid.

By way of example, non-limiting mention is made of the cocoamphodiacetate sold under the trade name MIRANOL C2M concentrate by the company Rhone Poulenc.

As non-limiting examples of non-silicone non-ionic surfactant(s), that may be used according to the present disclosure, mention may be made of the non-ionic surfactants that are well known per se (see, for example, “Handbook of Surfactants” by M. R. Porter, publishers Blackie & Son (Glasgow and London), 1991, pp 116-178). As non-limiting examples of such non-silicone non-ionic surfactants, mention may be made of alcohols, alpha-diols, alkylphenols or polyethoxylated, polypropoxylated or polyglycerolated fatty acids having a fatty chain containing, for example, from 8 to 18 carbon atoms, wherein the number of ethylene oxide or propylene oxide groups may, in one embodiment, range from 2 to 50 and the number of glycerol groups from 2 to 30. Non-limiting mention may also be made of copolymers of ethylene oxide and of propylene oxide, condensates of ethylene oxide and of propylene oxide with fatty alcohols; polyethoxylated fatty amides, including in at least one embodiment those having from 2 to 30 mol of ethylene oxide, polyglcyerolated fatty amides comprising, on average, 1 to 5 glycerol groups, for instance from 1.5 to 4 glycerol groups; polyethoxylated fatty amines, including in at least one embodiment, those having 2 to 30 mol of ethylene oxide; oxyethylenated sorbitan fatty acid esters having from 2 to 30 mol of ethylene oxide; sucrose fatty acid esters, polyethylene glycol fatty acid esters, alkylpolyglycosides, N-alkylglucamine derivatives, amine oxides, including, for example (C₁₀C₁₄)alkylamine oxides and N-acylaminopropylmorpholine oxides. According to at least one embodiment of the invention, the non-ionic surfactant is chosen from alkylpolyglycosides.

The non-silicone cationic surfactants that can be used may be chosen from surfactants that are well known per se including, for example, salts of primary, secondary or tertiary fatty amines, which are optionally polyoxyalkylenated, quaternary ammonium salts, and mixtures thereof.

As non-limiting examples of quaternary ammonium salts, mention may be made of:

quaternary ammonium salts of the formula: 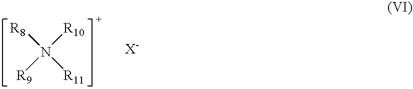
wherein:

radicals R8 to R11, which may be identical or different, are each independently chosen from linear or branched aliphatic radicals containing from 1 to 30 carbon atoms, and aromatic radicals, for example aryl or alkylaryl. The aliphatic radicals may contain heteroatoms such as, for example, oxygen, nitrogen, sulphur and/or halogens. The aliphatic radicals are, for example, chosen from alkyl, alkoxy, (C₂-C₆)polyoxyalkylene, alkylamide, (C₁₂-C₂₂)alkylamido(C₂-C₆)alkyl, (C₁₂-C₂₂)alkyl acetate, and hydroxyalkyl radicals containing from 1 to 30 carbon atoms; and X is an anion chosen from the group of halides, phosphates, acetates, lactates, (C₂-C₆)alkyl sulphates, alkyl sulphonates or alkylaryl sulphonates;

quaternary ammonium salts of imidazoline, for example those of the formula: 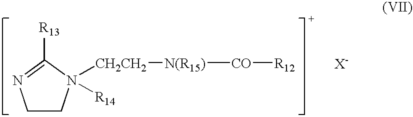
wherein:

R12 is chosen from an alkenyl or alkyl radical containing from 8 to 30 carbon atoms, for example fatty acid derivatives of tallow,

R13 is chosen from a hydrogen atom, a C₁-C₄ alkyl radical or an alkenyl or alkyl radical containing from 8 to 30 carbon atoms,

R14 is a C₁-C₄ alkyl radical,

R15 is chosen from a hydrogen atom or a C₁-C₄ alkyl radical,

X- is an anion chosen from the group of halides, phosphates, acetates, lactates, alkyl sulphates, alkyl sulphonates or alkylaryl sulphonates.

In at least one embodiment, R12 and R13 are each chosen from mixtures of alkenyl or alkyl radicals containing from 12 to 21 carbon atoms, for example fatty acid derivatives of tallow, R14 is a methyl radical, and R15 is a hydrogen atom. Such a quaternary ammonium salt is, for example, sold under the name Rewoquat® W 75 by the company Rewo;

the diquaternary ammonium salts of formula (VIII): 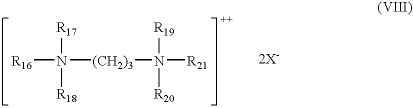
wherein:

R16 is chosen from aliphatic radicals containing from 16 to 30 carbon atoms,

R17, R18, R19, R20 and R21, which may be identical or different, are each independently chosen from hydrogen and alkyl radicals containing from 1 to 4 carbon atoms, and

X is an anion chosen from halides, acetates, phosphates, nitrates and methyl sulphates.

In at least one embodiment, the diquaternary ammonium salt is propanetallowdiammonium dichloride;

quaternary ammonium salts containing at least one ester function, including, for example, those of formula (IX) below: 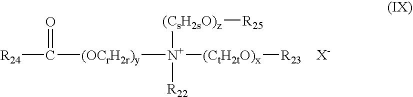
wherein:

R22 is chosen from C₁-C₆ alkyl radicals, C₁-C₆ hydroxyalkyl radicals, and C₁-C₆ dihydroxyalkyl radicals;

R23 is chosen from:
the radical 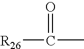
linear or branched, saturated or unsaturated C₁-C₂₂ hydrocarbon-based radicals R27, and
a hydrogen atom,

R25 is chosen from:
the radical 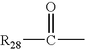
linear or branched, saturated or unsaturated C₁-C₆ hydrocarbon-based radicals R29, and
a hydrogen atom,

R24, R26 and R28, which may be identical or different, are each independently chosen from linear or branched, saturated or unsaturated C₇-C₂₁ hydrocarbon-based radicals;

r, s and t, which may be identical or different, are each independently chosen from integers ranging from 2 to 6;

y is an integer ranging from 1 to 10;

x and z, which may be identical or different, are each independently chosen from integers ranging from 0 to 10;

X- is a simple or complex, organic or inorganic anion;

with the proviso that the sum x+y+z ranges from 1 to 15, when x is 0, R23 denotes R27 and when z is 0, R25 denotes R29.

The alkyl radicals R22 may be linear or branched. In at least one embodiment, the alkyl radicals R22 are linear.

In at least one embodiment R22 is chosen from methyl, ethyl, hydroxyethyl and dihydroxypropyl radicals, for example a methyl or ethyl radical.

In at least one embodiment, the sum x+y+z ranges from 1 to 10.

In at least one embodiment, when R23 is a hydrocarbon-based radical R27, R23 may be long and contain from 12 to 22 carbon atoms, or may be short and contain from 1 to 3 carbon atoms.

In at least one embodiment, when R25 is a hydrocarbon-based radical R29, it contains from 1 to 3 carbon atoms.

In another non-limiting embodiment, R24, R26 and R28, which may be identical or different, are independently chosen from linear or branched, saturated or unsaturated C₁₁-C₂₁ hydrocarbon-based radicals, for example linear or branched, saturated or unsaturated C₁₁-C₂₁ alkyl and alkenyl radicals.

In another non-limiting embodiment, x and z, which may be identical or different, are independently chosen from 0 or 1.

In a further non-limiting embodiment, y is equal to 1.

In another non-limiting embodiment, r, s and t, which may be identical or different, are each independently chosen from 2 or 3, for example, 2.

The anion X- may be a halide (chloride, bromide or iodide) or an alkyl sulphate, (e.g., methyl sulphate). X- may also be chosen from methanesulphonate, phosphate, nitrate, tosylate, an anion derived from an organic acid such as acetate or lactate, or any other anion compatible with an ammonium containing an ester function.

In at least one embodiment, the anion X- is chloride or methyl sulphate.

In a non-limiting embodiment of the composition according to the present disclosure, ammonium salts of formula (IX) are used, in which:

R22 is chosen from a methyl or ethyl radical;

x and y are equal to 1;

z is equal to 0 or 1;

r, s and t are equal to 2;

R23 is chosen from:
the radical 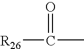
methyl, ethyl or C₁₄-C₂₂ hydrocarbon-based radicals, and
a hydrogen atom;

R25 is chosen from:
the radical 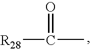
   and
a hydrogen atom;

R24, R26 and R28, which may be identical or different, are each independently chosen from linear or branched, saturated or unsaturated C₁₃-C₁₇ hydrocarbon-based radicals, for instance, from linear or branched, saturated or unsaturated C₁₃-C₁₇ alkyl and alkenyl radicals.

In at least one non-limiting embodiment of the present invention, the hydrocarbon-based radicals are linear.

As non-limiting examples of compounds of formula (IX), mention may be made of diacyloxyethyldimethylammonium, diacyloxyethylhydroxyethylmethylammonium, monoacyloxyethyidihydroxyethylmethylammonium, triacyloxyethylmethylammonium and monoacyloxyethylhydroxyethyldimethylammonium salts (for example, chloride or methyl sulphate), and mixtures thereof. In a non-limiting embodiment, the acyl radicals have from 14 to 18 carbon atoms and originate from a plant oil, such as palm oil or sunflower oil. If the compound contains several acyl radicals, these radicals may be identical or different.

The compounds of formula (IX) may be obtained, for example, by direct esterification of optionally oxyalkylenated triethanolamine, triisopropanolamine, alkyldiethanolamine or alkyldiisopropanolamine with fatty acids or with mixtures of fatty acids of plant or animal origin, or by transesterification of their methyl esters. This esterification is followed by a quaternization using an alkylating agent such as an alkyl (e.g., methyl or ethyl) halide, a dialkyl (e.g., dimethyl or diethyl) sulphate, methyl methanesulphonate, methyl para-toluenesulphonate, or glycol or glycerol chlorohydrin.

Such compounds are, for example, sold under the names Dehyquart® by the company Henkel, Stepanquat® by the company Stepan, Noxamium® by the company Ceca, and Rewoquat® WE 18 by the company Rewo-Witco.

As cationic surfactants, mixtures of the quaternary ammonium salts of mono-, di- and triesters with a weight majority of diester salts may be used in at least one embodiment.

Mixtures of ammonium salts that may be used include, for example, the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, wherein the acyl radicals contain from 14 to 18 carbon atoms and are derived from palm oil that is optionally partially hydrogenated.

Ammonium salts containing at least one ester function may also be used. Non-limiting examples of such ammonium salts are described in U.S. Pat. Nos. 4,874,554 and 4,137,180.

In at least one embodiment, the quaternary ammonium salts of formula (VI) are chosen from:
tetraalkylammonium chlorides, for example dialkyldimethylammonium chlorides or alkyltrimethylammonium chlorides, wherein the alkyl radical contains from 12 to 22 carbon atoms. Non-limiting examples of these tetralkylammonium chlorides include behenyltrimethylammonium chloride, distearyldimethylammonium chloride, cetyltrimethylammonium chloride and benzyldimethylstearylammonium chloride; and
palmitylamidopropyltrimethylammonium chloride and stearamidopropyldimethyl(myristyl acetate)ammonium chloride sold under the name Ceraphyl® 70 by the company Van Dyk.

In at least one embodiment, the cationic surfactants are chosen from quaternary ammonium salts, including, for example, behenyltrimethylammonium chloride and palmitylamidopropyltrimethylammonium chloride.

The non-silicone surfactants may be present in the emulsion of the present disclosure in an amount ranging from 0.1% to 30% by weight of active material; for example, from 0.5% to 15% by weight, relative to the total weight of the composition.

Non-limiting examples of surfactants that may be used in the present disclosure include, for example the silicone surfactants described in French patent FR 2 818 902. Silicone surfactants that may be used in the presently disclosed compositions are those that are well known to those skilled in the art. These surfactants may be water-soluble, spontaneously water-dispersible or water-insoluble. In at least one non-limiting embodiment, the surfactants are chosen from water-soluble or spontaneously water-dispersible surfactants.

The silicone surfactants may, for example, be chosen from the compounds of formulae (I)-(V) below: 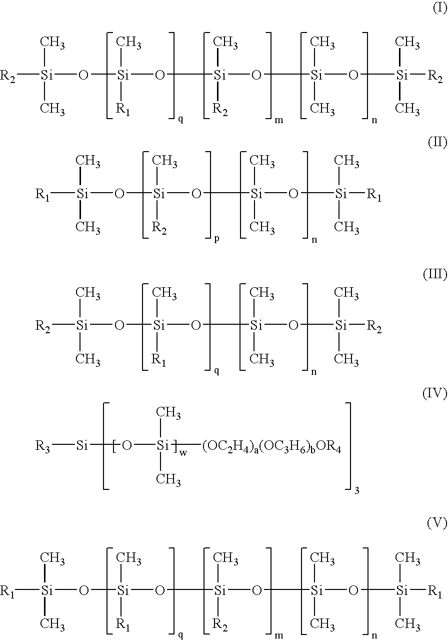
wherein:

R1, which may be identical or different, is chosen from linear or branched C₁-C₃₀ alkyl groups and phenyl groups;

R2, which may be identical or different, is chosen from —C_{c}H_{2c}—O—(C₂H₄O)ₐ—(C₃H₆O)_{b}—R5 and —C_{c}H_{2c}—O—(C₄H₈O)ₐ—R5;

R3 and R4, which may be identical or different, are each independently chosen from linear or branched C₁-C₁₂ alkyl groups, for example, a methyl group;

R5, which may be identical or different, is chosen from a hydrogen atom, a linear or branched alkyl group containing from 1 to 12 carbon atoms, a linear or branched alkoxy group containing from 1 to 6 carbon atoms, a linear or branched acyl group containing from 2 to 12 carbon atoms, a hydroxyl group, —SO₃M, —OCOR6, a C₁-C₆ aminoalkoxy optionally substituted on the amine, a C₂-C₆ aminoacyl optionally substituted on the amine, —NHCH₂CH₂COOM, —N(CH₂CH₂COOM)₂, a C₁-C₁₂ aminoalkyl optionally substituted on the amine and on the alkyl chain, a C₁-C₃₀ carboxyacyl, a phosphono group optionally substituted with one or two substituted C₁-C₁₂ aminoalkyl groups, —CO(CH₂)_{d}COOM, —OCOCHR7(CH₂)_{d}COOM, —NHCO(CH₂)_{d}OH, and —NH₃Y;

M, which may be identical or different, is chosen from a hydrogen atom, Na, K, Li, NH₄ and an organic amine;

R6 is a linear or branched C₁-C₃₀ alkyl group,

R7 is a hydrogen atom or an SO₃M group;

d ranges from 1 to 10;

m ranges from 0 to 20;

n ranges from 0 to 500;

p ranges from 1 to 50;

q ranges from 0 to 20;

a ranges from 0 to 50;

b ranges from 0 to 50;

a+b is greater than or equal to 1;

c ranges from 0 to 4;

w ranges from 1 to 100;

Y is a monovalent inorganic or organic anion, for example a halide (e.g., chloride, bromide), a sulphate or a carboxylate (e.g., acetate, lactate, citrate).

In at least one embodiment of the present disclosure, silicone surfactants corresponding to formula (IV) or (VII) as defined above are used. In another non-limiting embodiment, the composition of the present disclosure uses silicone surfactants corresponding to formula (IV) or (VII) in which at least one, and in some embodiments all, of the following conditions are met:

c is equal to 2 or 3;

R1 is a methyl group;

R5 is chosen from a hydrogen atom, a methyl group and an acetyl group, and in at least one embodiment, R5 is hydrogen;

a ranges from 1 to 25, for example from 2 to 25;

b ranges from 0 to 25, for example from 10 to 20;

n ranges from 0 to 100; and

p ranges from 1 to 20.

The silicone surfactants may, for example, be chosen from those sold under the trade names FLUID DC 193 and DC 5225C by the company Dow Corning, Silwet® L 77 by the company OSI and Mazil® 756 by the company Mazer PPG, and the mixture of Lauryl PEG/PPG-18/18 Methicone (and) Poloxamer 407 (and) Dodecene sold by Dow Corning under the name DC 5200.

Any type of ionicity can be used for the emulsions of the present disclosure. However, surfactants having an HLB (hydrophilic lipophilic balance) of less than 10, for example from 1.5-10, or from 1.5-7 are used in at least one embodiment of the present disclosure. For the purpose of the present disclosure, HLB or hydrophilic-lipophilic balance of the non-ionic surfactant(s) used according to the present disclosure is the HLB according to Griffin defined in the publication J. Soc. Cosm. Chem. 1954 (Volume 5), pages 249-256.

The at least one surfactant may be present in the composition in an amount ranging from 0.01% to 30% by weight, for example from 0.1% to 30% by weight, e.g., from 0.2% to 15% by weight, relative to the total weight of the emulsion.

In a non-limiting embodiment of the present disclosure, the at least one surfactant present in the emulsion is chosen from silicone surfactants.

The silicone surfactants may be present in the emulsion of the present disclosure in an amount ranging from 0.1% to 30% by weight, for example from 0.2% to 15% by weight, relative to the total weight of the emulsion.

The at least one cyanoacrylate monomer present in the composition of the present disclosure may be chosen, in at least one embodiment, from monomers of formula (IX):

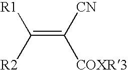

wherein:
X is chosen from NH, S or O,
R1 and R2, which may be the same or different, are each independently chosen from sparingly electron-withdrawing or non-electron-withdrawing groups (sparingly inductive-withdrawing or non-inductive-withdrawing groups) for example:

a hydrogen atom,

a saturated or unsaturated, linear, branched or cyclic hydrocarbon-based group, containing, in at least one embodiment, from 1 to 20 carbon atoms, such as from 1 to 10 carbon atoms, optionally containing at least one atom chosen from nitrogen, oxygen and sulphur atoms, and optionally substituted with at least one group chosen from —OR, —COOR, —COR, —SH, —SR, —OH, and halogen atoms,

a modified or unmodified polyorganosiloxane residue,

a polyoxyalkylene group,

R is chosen from saturated or unsaturated, linear, branched or cyclic hydrocarbon-based groups containing, in at least one embodiment, from 1 to 20 carbon atoms, such as from 1 to 10 carbon atoms, optionally containing at least one atom chosen from nitrogen, oxygen and sulphur atoms, and optionally substituted with at least one group chosen from —OR′, —COOR′, —COR′, —SH, —SR′, —OH, and halogen atoms, and from a polymer residue that may be obtained by free-radical polymerization, by polycondensation or by ring opening, R′ being chosen from C₁-C₁₀ alkyl groups.

As used herein, the terms “electron-withdrawing group,” or “inductive-withdrawing group” mean any group that is more electronegative than carbon. Reference is made to the publication PR Wells Prog. Phys. Org. Chem., Vol 6, 111 (1968).

As used herein, the term “sparingly electron-withdrawing or non-electron-withdrawing group” means any group whose electronegativity is less than or equal to that of carbon.

In a non-limiting embodiment of the present disclosure, the alkenyl or alkynyl groups disclosed above contain 2 to 20 carbon atoms, for example, from 2 to 10 carbon atoms.

With respect to the saturated or unsaturated, linear, branched or cyclic hydrocarbon groups, mention may be made, for example, of C₁-C₂₀ linear or branched alkyl, alkenyl or alkynyl groups, such as methyl, ethyl, n-butyl, tert-butyl, isobutyl, pentyl, hexyl, octyl, butenyl or butynyl; cycloalkyl or aromatic groups.

As substituted hydrocarbon-based groups, non-limiting mention may be made of hydroxyalkyl or polyhaloalkyl groups.

With respect to the modified and unmodified polyorganosiloxane residues, non-limiting examples of unmodified polyorganosiloxanes include polyalkylsiloxanes such as polydimethylsiloxanes, polyarylsiloxanes such as polyphenylsiloxanes, and polyarylalkylsiloxanes such as polymethylphenylsiloxanes. Non-limiting examples of modified polyorganosiloxanes include polydimethylsiloxanes containing polyoxyalkylene, siloxy, silanol, amine, imine and/or fluoroalkyl groups, and mixtures thereof.

With respect to the polyoxyalkylene groups, non-limiting mention may be made of polyoxyethylene groups and polyoxypropylene groups, containing, in at least one embodiment, from 1 to 200 oxyalkylene units.

Among the mono- or polyfluoroalkyl groups disclosed for R3 and R4, non-limiting mention may be made of —(CH₂)ₙ—(CF₂)ₘ—CF₃ or —(CH₂)ₙ—(CF₂)ₘ—CHF₂, wherein n ranges from 1 to 20 and m ranges from 1 to 20.

The substituents R1 and R2 may be optionally substituted with a group having a cosmetic activity. For example, R1 and R2 may be substituted with groups having coloring, antioxidant, UV-screening and conditioning functions.

Non-limiting examples of groups with a coloring function include azo, quinone, methine, cyanomethine and triarylmethane groups.

Non-limiting examples of groups with an antioxidant function include, butylhydroxyanisole (BHA), butylhydroxytoluene (BHT) and vitamin E type groups.

Non-limiting examples of groups with a UV-screening function include benzophenone, cinnamate, benzoate, benzylidenecamphor and benzoylmethane type groups.

Non-limiting examples of groups with a conditioning function include cationic groups of the fatty ester type.

In at least one non-limiting embodiment, R1 and R2 are each a hydrogen atom, R′3 is chosen from a hydrogen atom and a saturated or unsaturated, linear, branched or cyclic hydrocarbon-based group, wherein said hydrocarbon-based group contains, in at least one embodiment, from 1 to 20 carbon atoms, e.g., from 1 to 10 carbon atoms, optionally contains at least one atom chosen from nitrogen, oxygen and sulphur atoms, and is optionally substituted with at least one group chosen from —OR′, —COOR′, —COR′, —SH, —SR′, —OH, and halogen atoms, or a polymer residue that may be obtained by free-radical polymerization, by polycondensation or by ring opening, wherein R′ is chosen from C₁-C₁₀ alkyl groups.

In at least one non-limiting embodiment, R′3 is chosen from saturated hydrocarbon-based groups containing from 1 to 10 carbon atoms.

In at least one non-limiting embodiment, X is an oxygen atom.

As non-limiting examples of compounds of formula (IX), mention may be made of the monomers:

a) belonging to the family of polyfluoroalkyl 2-cyanoacrylates, for example: the ester 2,2,3,3-tetrafluoropropyl 2-cyano-2-propenoate of formula:

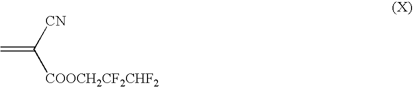

or the ester 2,2,2-trifluoroethyl 2-cyano-2-propenoate of formula:

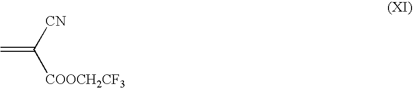

b) the alkyl or alkoxyalkyl 2-cyanoacrylates 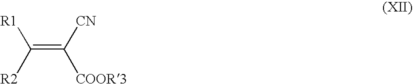
wherein:

R′b 3 is chosen fom C₁-C₁₀ alkyl, (C₁-C₄)alkoxy(C₁-C₁₀)alkyl or C₂-C₁₀ alkenyl radicals.

Further non-limiting mention may be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

In at least one non-limiting embodiment, the alkyl or alkoxyalkyl 2-cyanoacrylate monomers of formula XII above are used. In a further non-limiting embodiment, the cyanoacrylate monomer(s) is (are) chosen from C₆-C₁₀ alkyl cyanoacrylates.

In at least one embodiment, the monomers are chosen from the octyl cyanoacrylates of formula XIII, and mixtures thereof:

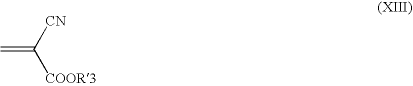

wherein:
R′3 is chosen from:
   —(CH₂)₇—CH₃,
   —CH(CH₃)—(CH₂)₅—CH₃,
   —CH₂—CH(C₂H₅)—(CH₂)₃—CH₃,
   —(CH₂)₅—CH(CH₃)—CH₃, and
   —(CH₂)₄—CH(C₂H₅)—CH₃.

The monomers used in accordance with the present disclosure may be covalently bonded to at least one support, such as polymers, oligomers or dendrimers. The polymer or the oligomer can be in linear, branched, comb or block form. The distribution of the monomers of the present disclosure over the polymeric, oligomeric or dendritic structure may be random, in an end position or in the form of blocks.

The cyanoacrylate monomer(s) may be present in an amount ranging from 0.1% to 80% by weight, such as from 0.2% to 60% by weight, for example from 0.5% to 50% by weight, relative to the total weight of the cosmetic composition.

In a non-limiting embodiment of the present disclosure, the electrophilic monomers are chosen from monomers that are capable of polymerizing anionically in the presence of a nucleophilic agent. As used herein, the term “anionic polymerization,” means the mechanism defined in the publication “Advanced Organic Chemistry”, 3^{rd} Edition by Jerry March, pages 151 to 161.

Nucleophilic agents capable of initiating anionic polymerization are systems that are known in themselves, and are capable of generating a carbanion on contact with a nucleophilic agent, such as the hydroxyl ions contained in water. As used herein, the term “carbanion” means the chemical species defined in “Advanced Organic Chemistry”, 3^{rd} Edition by Jerry March, page 141.

The nucleophilic agent of the present disclosure is a molecular compound, an oligomer, a dendrimer or a polymer that has nucleophilic functions. Non-limiting examples of nucleophilic functions include the following functions: R₂N⁻, NH₂⁻, Ph₃C⁻, R₃C⁻, PhNH⁻, pyridine, ArS⁻, R—C^{≡}C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group; Ar is an aryl group and R is a C₁-C₁₀ alkyl group.

The electrophilic monomers may be synthesized according to known methods described in the art. For example, the cyanoacrylate monomers may be synthesized according to the disclosure of the following U.S. Pat. Nos. 3,527,224, 3,591,767, 3,667,472, 3,995,641, 4,035,334 and 4,650,826.

In at least one embodiment of the present disclosure, the monomers are chosen from monomers capable of polymerizing on keratin fibers under acceptable cosmetic conditions. For example, polymerization of the monomer may be performed at a temperature of less than or equal to 80° C., which does not prevent the application from being completed by drying under a drying hood, blow-drying or treating with a flat iron or a crimping iron.

The composition of the present disclosure may also contain one or more polymerization inhibitors, for example anionic and/or free-radical polymerization inhibitors, in order to increase the stability of the composition over time. As non-limiting examples of these polymerization inhibitors, mention may be made of: sulphur dioxide, nitric oxide, boron trifluoride, hydroquinone and derivatives thereof, for example hydroquinone monoethyl ether, TBHQ, benzoquinone and derivatives thereof, for example duroquinone, catechol and derivatives thereof, for example t-butyl catechol and methoxycatechol, anisole and its derivatives, for example methoxyanisole or hydroxyanisole, pyrogallol and derivatives thereof, p-methoxyphenol, hydroxybutyltoluene, alkyl sulphates, alkyl sulphites, alkyl sulphones, alkyl sulphoxides, alkyl sulphides, mercaptans, 3-sulphonene, and mixtures thereof. In at least one embodiment of the present disclosure, the alkyl groups are chosen from groups having from 1 to 6 carbon atoms.

Inorganic or organic acids may also be used as inhibitors.

Thus, the cosmetic composition according to the present disclosure may also comprise at least one inorganic or organic acid, the latter comprising at least one carboxylic or sulphonic group and having a pKa ranging from 0 to 6, such as phosphoric acid, hydrochloric acid, nitric acid, benzenesulphonic acid, toluenesulphonic acid, sulphuric acid, carbonic acid, hydrofluoric acid, acetic acid, formic acid, propionic acid, benzoic acid, mono-, di- or trichloroacetic acids, salicylic acid, trifluoroacetic acid, octanoic acid, heptanoic acid and hexanoic acid.

In a non-limiting embodiment of the present disclosure, acetic acid is the organic acid.

The concentration of inhibitor in the cosmetic composition of the present disclosure may range from 10 ppm to 30% by weight, for example from 10 ppm to 15% by weight, relative to the total weight of the composition.

To modify the anionic polymerization kinetics, it is possible to increase the nucleophilicity of keratin fibers via chemical conversion of the keratin material.

One non-limiting example of such chemical conversion is the reduction of the disulphide bridges of which keratin is partly composed into thiols before applying the composition of the invention. Non-limiting examples of reducing agents suitable for reducing the disulphide bridges of which keratin is partly composed include:

anhydrous sodium thiosulphate,

powdered sodium metabisulphite,

thiourea,

ammonium sulphite,

thioglycolic acid,

thiolactic acid,

ammonium thiolactate,

glyceryl monothioglycolate,

ammonium thioglycolate,

thioglycerol,

2,5-dihydroxybenzoic acid,

diammonium dithioglycolate,

strontium thioglycolate,

calcium thioglycolate,

zinc formosulphoxylate,

isooctyl thioglycolate,

dl-cysteine, and

monoethanolamine thioglycolate.

The water-in-oil emulsion of the present disclosure may also comprise at least one agent that is normally used in cosmetics, for example, reducing agents, fatty substances, plasticizers, softeners, antifoams, moisturizers, pigments, clays, inorganic fillers, UV-screening agents, inorganic colloids, peptizers, solubilizing agents, fragrances, preserving agents, fixing or non-fixing polymers, polyols, proteins, vitamins, direct or oxidation dyes, pearlescent agents, propellants, inorganic or organic thickeners, oxyethylenated or nonoxyethylenated waxes, paraffins, C₁₀-C₃₀ fatty acids, such as stearic or lauric acid, C₁₀-C₃₀ fatty amides, such as lauric diethanolamide, C₁₀-C₃₀ fatty alcohol esters such as C₁₀-C₃₀ fatty alcohol benzoates, and mixtures thereof.

In at least one embodiment of the present disclosure, the emulsion comprises at least one cosmetic additive chosen from pigments, nano/micro objects, liquid crystals, oxidizing agents, direct dyes, fluorescent dyes, oxidation dyes, polymers and reducing agents.

The direct dyes may, for example, be chosen from nitrobenzene dyes, azo direct dyes, methine direct dyes, or hydrazone dyes, and may be cationic or non-cationic.

The oxidation dyes are oxidation bases and couplers that are well known in the art. As examples of couplers, non-limiting mention may be made of meta-phenylenediamines, meta-aminophenols, meta-diphenols, naphthalene couplers, heterocyclic couplers and the addition salts thereof. As examples of oxidation bases, non-limiting mention may be made of para-phenylenediamines, bisphenylalkylenediamines, para-aminophenols, ortho-aminophenols and heterocyclic bases.

The pigments that may be used are known in the art, and are described in the Encyclopedia of Chemical Technology by Kirk-Othmer and in the Encyclopaedia of Industrial Chemistry by Ullmann.

These pigments may be in the form of a pigmentary powder or paste. The pigments may, for example, be chosen from white or colored pigments, lakes, special-effect pigments such as pearlescent agents or flakes, and mixtures thereof.

As used herein, the term “polymer” means any of the natural or synthetic polymers that can be used in cosmetics, including polymers obtained by free-radical or anionic polymerization, by polycondensation or by ring opening. These polymers may be linear, branched or star polymers.

In at least one embodiment of the present disclosure, natural polymers are used. These polymers may or may not be chemically modified, and may, for example, be chosen from dextrans, celluloses (carboxymethylhydroxypropylcelluloses), guars (carboxymethylhydroxypropylguars), starches, alginates and chitosans.

The pigments and the fillers may be coated with organic or inorganic compounds.

The pigments and fillers may be coated with fatty substances including, for example, parleam, silicone compounds, fatty acids, or fatty alcohols, for example palmitic acid, palmityl alcohol, stearic acid and stearyl alcohol, and mixtures thereof. The fatty acids may be in the form of sodium, potassium, magnesium, iron, titanium, zinc or aluminium salts.

According to at least one embodiment, the aqueous phase of the emulsion of the present disclosure comprises a water-soluble cosmetic active agent, i.e., which is soluble in the aqueous phase. As examples of water-soluble cosmetic active agents, non-limiting mention is made of oxidizing agents, direct dyes, fluorescent dyes, oxidation dyes, polymers and reducing agents.

The water-in-oil emulsion of the present disclosure may be formulated in various galenic forms, including a lotion, an aerosol foam, a conditioner, a shampoo, a gel, or a wax.

The water-in-oil emulsion of the present disclosure is a cosmetic emulsion. As used herein, the term “cosmetic emulsion,” means an emulsion compatible with keratin materials.

It is possible to prepare several emulsions of the water-in-oil (W/O) type of different compositions and to mix them in varying proportions so as to obtain a new emulsion containing various cosmetic active agents.

The process of the present disclosure comprises the application of the water-in-oil emulsion described above to keratin fibers. In this case, it is possible to optionally mix, at the time of use, with the emulsion of the invention, a composition optionally containing other cosmetic active agents in a cosmetic medium. The composition/mixture is then applied to dry hair or hair that has been previously moistened or shampooed.

The hair is dried with a hairdryer, hood or iron. The hair may optionally be shampooed again after drying.

The emulsion of the present disclosure may, for example, be used for bleaching keratin fibers. In this case, the emulsion itself may contain an oxidizing agent or the oxidizing agent may be added extemporaneously at the time of use. Non-limiting examples of oxidizing agents that may be used include hydrogen peroxide, urea peroxide, alkali metal perbromates, persalts, for example perborates and persulphates, peracids and oxidase enzymes, for example peroxidases, 2-electron oxidoreductases (such as uricases), and 4-electron oxygenases (such as laccases).

The emulsion of the present disclosure may be used for oxidation dyeing. In this case, the emulsion may contain an oxidizing agent or an oxidation dye precursor. A composition comprising the oxidation dye and/or a composition comprising the oxidizing agent may be added to the emulsion of the present disclosure at the time of use.

The emulsion of the present disclosure may be used for direct dyeing. In this case, the direct dye may be present in the emulsion of the present disclosure or may be added to the emulsion at the time of use. In the case of direct lightening dyeing, the emulsion of the present disclosure may contain the direct dye and the oxidizing agent. In a non-limiting embodiment of the present disclosure, the direct dye and/or the oxidizing agent is (are) added to the emulsion of the present disclosure at the time of use.

The emulsion of the present disclosure may be used for permanent-waving or thiol hair straightening. In this case, the emulsion may comprise at least one reducing agent, for example thioglycolic acid or cysteine. The reducing agent may be added to the emulsion at the time of use.

The emulsion of the present disclosure may be used for alkaline hair straightening. In this case, the emulsion may contain one or more inorganic bases, for example sodium hydroxide or lithium hydroxide, or an organic base chosen from guanidinium salts. These bases may also be added to the emulsion at the time of use.

In at least one embodiment, the hair is pretreated with at least one nucleophilic agent. The hair is then optionally dried, and the emulsion is then applied to the hair. The hair is then dried (via a hairdryer, hood or iron) and optionally shampooed again.

In another non-limiting embodiment, the hair is pretreated with at least one cosmetic additive other than a nucleophilic agent. The hair is optionally dried, and the emulsion of the invention is then applied to the hair. The hair is then dried (via a hairdryer, hood or iron) and optionally shampooed again.

In at least one embodiment of the present disclosure, the process of the disclosure comprises combining the application of the emulsion of the invention with treatments on the fiber, such as stripping the fiber and/or mordanting the fiber, in order to render the treatment more effective.

According to this embodiment, at least one permanent-wave reducing agent, permanent-waving product, oxidation dyeing product, bleaching product, shampooing product, hairstyling product or alkaline hair straightening product may be applied to the fiber as a pretreatment, followed by the application of one of the processes described above.

In another non-limiting embodiment, the emulsion of the present disclosure is applied as a pretreatment in order to protect the keratin fiber before the application of treatments known to be capable of degrading the hair. It is then possible to optionally remove the makeup from the fiber.

Thus, after having applied the composition of the present disclosure according to one of the processes already described, a permanent-wave reducing agent, permanent-waving agent, oxidation dyeing agent, bleaching agent, or alkaline hair straightening agent is applied.

The application of the emulsion of the disclosure may also be carried out after oxidation or direct dyeing, or after the application of a mixture of these dyes, in order to protect the brightness of the color against shampooing.

The present disclosure also relates to a kit comprising a first emulsion as described above and a second composition comprising the nucleophilic agent.

The present disclosure also relates to a kit comprising a first composition containing at least one cyanoacrylate monomer and at least one anionic and/or free-radical polymerization inhibitor and/or an acid, and a second composition containing a water-in-oil emulsion comprising an aqueous phase, an oily phase and a surfactant, and optionally an organic acid.

The process of the present diclosure may be carried out several times in order to perform multiple applications of the emulsion of the disclosure so as to obtain a superimposition of layers in order to attain specific coating properties in terms of chemical nature, mechanical strength, thickness, appearance and feel.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature.

### EXAMPLES

### Example 1

An emulsion was obtained from the following composition:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | 3 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Water | 20 | g |
| Acetic acid | 1 | g |
| Methylheptyl cyanoacrylate from Chemence | 11.5 | g |

0.5 g of this emulsion was applied to a 1 g lock of chestnut brown hair that was previously shampooed. After a waiting period of 15 min at ambient temperature, the lock was dried with a hairdryer for 3 min. A final shampooing procedure was carried out. The hair felt soft and tangle-free. There were no unpleasant residues. This effect was still present after several shampooing procedures.

### Example 2

An emulsion was obtained from the following composition:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | 3.4 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Water | 20 | g |
| Carboxymethylhydroxypropylguar - Jaguar CMHP from Rhodia | 2 | g |
| Acetic acid | 1 | g |
| Methylheptyl cyanoacrylate from Chemence | 12 | g |

0.5 g of this emulsion was applied to a 1 g lock of chestnut brown hair that was previously shampooed. After a waiting period of 15 min at ambient temperature, the lock was dried with a hairdryer for 3 min. A final shampooing procedure was carried out. The hair was tangle-free and appeared to have more body. There were no unpleasant residues. This effect was still present after several shampooing procedures.

### Example 3

An emulsion was obtained from the following composition:

| | | |
|---|---|---|
| α,ω-dihydroxylated polydimethylsiloxane/ | 36 | g |
| cyclopentadimethylsiloxane (14.7/85.3) sold by Dow Corning | | |
| under the name DC 1501 Fluid | | |
| Cyclopentadimethylsiloxane sold by Dow Corning under the | 36 | g |
| name DC245 Fluid | | |
| Mixture of oxyethylenated oxypropylenated | 2.7 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Pearlescent agent Mica-titanium oxide coated with lithol B red | 10 | g |
| calcium salt lake*. | | |
| -Cellini Red 420 CR 7F from Engelhard- | | |
| Water | 16 | g |
| Acetic acid | 0.46 | g |
| Methylheptyl cyanoacrylate from Chemence | 11.5 | g |

*Pearlescent agent coated with polyisobutene and with palmitic acid.

0.5 g of this emulsion was applied to a 1 g lock of white hair that was previously shampooed. After a waiting period of 15 min at ambient temperature, the lock was dried with a hairdryer for 3 min. The lock was colored. The coloration obtained withstood shampooing. The lock obtained felt soft.

### Example 4

An emulsion was obtained from the following composition:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | | |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Dye: 3,6-bis(bis[biphenyl]methyl)amino)-2,5- | 10 | g |
| pyrazinedicarbonitrile | | |
| Water | 16 | g |
| Acetic acid | 0.46 | g |
| Methylheptyl cyanoacrylate from Chemence | 11.5 | g |

0.5 g of this emulsion was applied to a clean and wet lock of 1 g of natural chestnut brown hair having a tone depth of 4. After a waiting period of 15 min at ambient temperature, the lock was dried with a hairdryer for 3 minutes. The lock was colored. The coloration obtained withstood shampooing. The lock obtained felt soft.

### Example 5

An emulsion was obtained from the following composition:

| | | |
|---|---|---|
| α,ω-dihydroxylated polydimethylsiloxane/ | 36 | g |
| cyclopentadimethylsiloxane (14.7/85.3) sold by Dow Corning | | |
| under the name DC 1501 Fluid | | |
| Cyclopentadimethylsiloxane sold by Dow Corning under the | 36 | g |
| name DC245 Fluid | | |
| Mixture of oxyethylenated oxypropylenated | 2.7 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Pearlescent agent Mica-titanium oxide coated with lithol B red | 10 | g |
| calcium salt lake*. | | |
| -Cellini Red 420 CR 7F from Engelhard- | | |
| Water | 16 | g |
| Citric acid | 0.6 | g |
| Methylheptyl cyanoacrylate from Chemence | 11.5 | g |

*Pearlescent agent coated with polyisobutene and with palmitic acid.

0.5 g of this emulsion was applied to a 1 g lock of white hair that was previously shampooed. After a waiting period of 15 min at ambient temperature, the lock was dried with a hairdryer for 3 min. The lock was colored. The coloration obtained withstood shampooing. The lock obtained felt soft.

### Example 6

An emulsion was obtained from the following composition:

| | | |
|---|---|---|
| α,ω-dihydroxylated polydimethylsiloxane/ | 36 | g |
| cyclopentadimethylsiloxane (14.7/85.3) sold by Dow Corning | | |
| under the name DC 1501 Fluid | | |
| Cyclopentadimethylsiloxane sold by Dow Corning under the | 36 | g |
| name DC245 Fluid | | |
| Mixture of oxyethylenated oxypropylenated | 2.7 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Pearlescent agent Mica-titanium oxide coated with lithol B red | 10 | g |
| calcium salt lake*. | | |
| -Cellini Red 420 CR 7F from Engelhard- | | |
| Water | 16 | g |
| Citric acid | 0.6 | g |
| Methylheptyl cyanoacrylate from Chemence | 11.5 | g |

*Pearlescent agent coated with polyisobutene and with palmitic acid.

0.5 g of this emulsion was applied to a 1 g lock of white hair that was previously shampooed. After a waiting period of 15 min at ambient temperature, the lock was dried with a hairdryer for 3 min. The lock was colored. The coloration obtained withstood shampooing. The lock obtained felt soft.

### Example 7

An emulsion was obtained from the following composition:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | 3 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Behenyltrimethylammonium chloride | 0.1 | g |
| Water | 20 | g |
| Acetic acid | 1 | g |
| Methylheptyl cyanoacrylate from Chemence | 11.5 | g |

0.5 g of this emulsion was applied to a 1 g lock of chestnut brown hair that was previoously shampooed. After a waiting period of 15 min at ambient temperature, the lock was dried with a hairdryer for 3 min. A final shampooing procedure was carried out. The hair felt soft and tangle-free. There were no unpleasant residues. This effect was still present after several shampooing procedures.

### Example 8

A two-step application of an emulsion containing the electrophilic monomer and of a bleaching composition was carried out as follows.

The following compositions were prepared:

Emulsion A:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | 3 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Water | 20 | g |
| Citric acid | 0.6 | g |
| Methylheptyl cyanoacrylate from Chemence | 11.5 | g |

Composition B:

| | |
|---|---|
| 6% H₂O₂ | 50 g |
| 20.5% aqueous ammonia | 10 g |

0.5 g of composition B was applied to a 1 g lock of chestnut brown hair that was previously shampooed. After a waiting period of 15 min at ambient temperature, the lock was dried. Emulsion A was then applied. After a waiting period of 15 min at ambient temperature, the lock was dried with a hairdryer for 3 min. A final shampooing procedure was carried out. The hair was bleached, and felt soft and tangle-free. There were no unpleasant residues. This effect was still present after several shampooing procedures.

In another embodiment of this example, composition A was applied before composition B. The hair was bleached, and felt soft and tangle-free. There were no unpleasant residues. This effect was still present after several shampooing procedures.

### Example 9

A two-step application of an emulsion containing the electrophilic monomer and of a dyeing composition was carried out as follows.

The following composition was prepared:

Composition A:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 40 | g |
| name DC245 Fluid | | |
| Mixture of oxyethylenated oxypropylenated | 3 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Water | 20 | g |
| Citric acid | 0.6 | g |
| Methylheptyl cyanoacrylate from Chemence | 11.5 | g |

The dye composition known as Color Pulse® Red, which contains direct dyes, was applied to a 1 g lock of white hair that was previously. After a waiting period of 30 min at ambient temperature, the lock was shampooed and dried. Composition A was then applied. After a waiting period of 15 min at ambient temperature, the lock was dried with a hairdryer for 3 min. After 10 shampooing procedures were carried out on the lock, the color was much less washed-out than a lock not treated with composition A. Furthermore, hair treated with composition A felt soft and tangle-free. There were no unpleasant residues.

### Example 10

The following emulsion was prepared:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Cyclopentadimethylsiloxane sold by Dow Corning under the | 5 | g |
| name DC245 Fluid | | |
| Mixture of oxyethylenated oxypropylenated | 0.5 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Water | 83 | g |
| Acetic acid | 1.5 | g |
| Methylheptyl cyanoacrylate from Chemence | 10 | g |

0.5 g of this emulsion was applied to a 1 g lock of chestnut brown hair that was previously shampooed. After a waiting period of 15 min at ambient temperature, the lock was dried with a hairdryer for 3 min. A final shampooing procedure was carried out.

### Example 11

The following emulsion was prepared:

| | | |
|---|---|---|
| Mineral oil (paraffinum liquidum) | 7 | g |
| Hydroxyethyldiethonium polyisobutenyl | 1 | g |
| triethylaminosuccinate | | |
| Water | 84 | g |
| Acetic acid | 3 | g |
| Methylheptyl cyanoacrylate from Chemence | 5 | g |

0.5 g of this emulsion was applied to a 1 g lock of chestnut brown hair that was previously shampooed. After a waiting period of 15 min at ambient temperature, the lock was dried with a hairdryer for 3 min. A final shampooing procedure was carried out.

### Example 12

The following emulsion was prepared:

| | | |
|---|---|---|
| Mineral oil (paraffinum liquidum) | 5 | g |
| Olea Europa (olive) fruit oil/olea Europa | 2 | g |
| Hydroxyethyldiethonium polyisobutenyl | 1 | g |
| triethylaminosuccinate | | |
| Water | 84 | g |
| Acetic acid | 3 | g |
| Methylheptyl cyanoacrylate from Chemence | 5 | g |

0.5 g of this emulsion was applied to a 1 g lock of chestnut brown hair that was previously shampooed. After a waiting period of 15 min at ambient temperature, the lock was dried with a hairdryer for 3 min. A final shampooing procedure was carried out.

### Example 13

The following emulsion was prepared:

| | | |
|---|---|---|
| α,ω-dihydroxylated polydimethylsiloxane/ | 9.3 | g |
| cyclopentadimethylsiloxane (14.7/85.3) sold by Dow Corning | | |
| under the name DC 1501 Fluid | | |
| Cyclopentadimethylsiloxane sold by Dow Corning under the | 9.3 | g |
| name DC245 Fluid | | |
| Mixture of oxyethylenated oxypropylenated | 5.4 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Pearlescent agent Mica-titanium oxide coated with lithol B red | 2 | g |
| calcium salt lake*. | | |
| -Cellini Red 420 CR 7F from Engelhard- | | |
| Water | 63.9 | g |
| Methylheptyl cyanoacrylate from Chemence | 10 | g |

*Pearlescent agent coated with polyisobutene and with palmitic acid.

0.5 g of this emulsion was applied to a 1 g lock of white hair that was previously shampooed. After a waiting period of 15 min at ambient temperature, the lock was dried with a hairdryer for 3 min. The lock was colored. The coloration obtained withstood shampooing. The lock obtained felt soft.

### Example 14

The following emulsion was prepared:

| | | |
|---|---|---|
| α,ω-dihydroxylated polydimethylsiloxane/ | 40 | g |
| cyclopentadimethylsiloxane (14.7/85.3) sold by Dow Corning | | |
| under the name DC 1501 Fluid | | |
| Mixture of oxyethylenated oxypropylenated | 10 | g |
| polydimethylsiloxane (18 mol of ethylene oxide/18 mol of | | |
| propylene oxide), cyclopentadimethylsiloxane and water in the | | |
| proportions 10/88/2 - DC 5225C from Dow Corning | | |
| Pearlescent agent Mica-titanium oxide coated with lithol B red | 10 | g |
| calcium lake*. | | |
| -Cellini Red 420 CR 7F from Engelhard- | | |
| Water | 40 | g |
| Citric acid | 1 | g |
| Methylheptyl cyanoacrylate from Chemence | 11.4 | g |

*Pearlescent agent coated with polyisobutene and with palmitic acid.

0.5 g of this emulsion was applied to a 1 g lock of white hair that was previously shampooed. After a waiting period of 15 min at ambient temperature, the lock was dried with a hairdryer for 3 min. The lock was colored. The coloration obtained withstood shampooing. The lock obtained felt soft.

What is claimed is:

1. A water-in-oil emulsion for the treatment of keratin fibers, comprising an aqueous phase, at least one surfactant, at least one monomer polymerization inhibitor and an oily phase, wherein said oily phase comprises at least one cyanoacrylate monomer capable of polymerizing anionically in the presence of a nucleophilic agent.

2. The water-in-oil emulsion of claim 1, wherein the oily phase comprises at least one liquid organic solvent.

3. The water-in-oil emulsion of claim 2, wherein the at least one liquid organic solvent is chosen from aromatic alcohols; liquid fatty alcohols, polyols; volatile silicones; mineral, organic or plant oils; alkanes; liquid fatty acids and liquid fatty esters.

4. The water-in-oil emulsion of claim 2, wherein the at least one liquid organic solvent comprises a volatile silicone, alone or as a mixture with other silicones.

5. The water-in-oil emulsion of claim 1, wherein the at least one cyanoacrylate monomer is chosen from monomers of formula (I): 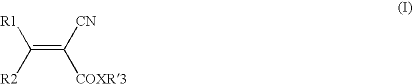
wherein:
X is chosen from NH, S and O,
R1 and R2 are each independently chosen from a sparingly electron-withdrawing or non-electron-withdrawing groups (sparingly inductive-withdrawing or non-inductive-withdrawing groups);
R′3 is chosen from a hydrogen atom and a saturated or unsaturated, linear, branched or cyclic hydrocarbon-based group, optionally comprising at least one atom chosen from nitrogen, oxygen and sulphur atoms, and optionally substituted with at least one group chosen from —OR′, —COOR′, —COR′, —SH, —SR′, —OH, and halogen atoms, and a polymer residue that is optionally obtained by free-radical polymerization, by polycondensation or by ring opening, R′ being chosen from C₁-C₁₀ alkyl groups.

6. The water-in-oil emulsion of claim 5, wherein in formula (I), R1 and R2 are each independently chosen from
a hydrogen atom,
a saturated or unsaturated, linear, branched or cyclic hydrocarbon-based group optionally containing at least one atom chosen from nitrogen, oxygen and sulphur atoms, and optionally substituted with at least one group chosen from —OR, —COOR, —COR, —SH, —SR, —OH, and halogen atoms,
a modified or unmodified polyorganosiloxane residue,
a polyoxyalkylene group,
wherein R is chosen from saturated or unsaturated, linear, branched or cyclic hydrocarbon-based groups optionally containing at least one atom chosen from nitrogen, oxygen and sulphur atoms, and optionally substituted with at least one group chosen from —OR′, —COOR′, —COR′, —SH, —SR′, —OH, and halogen atoms, and a polymer residue that may be obtained by free-radical polymerization, by polycondensation or by ring opening, R′ being chosen from C₁-C₁₀ alkyl groups.

7. The water-in-oil emulsion of claim 6, wherein the at least one cyanoacrylate monomer is chosen from the monomers of formula (IV): 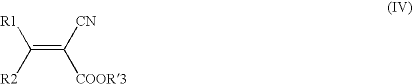
wherein R′3 is chosen from C₁-C₁₀ alkyl, C₂-C₁₀ alkenyl or (C₁-C₄)alkoxy(C₁-C₁₀)alkyl radicals and
wherein R1 and R2 are each independently chosen from
a hydrogen atom,
a saturated or unsaturated, linear, branched or cyclic hydrocarbon-based group optionally containing at least one atom chosen from nitrogen, oxygen and sulphur atoms, and optionally substituted with at least one group chosen from —OR, —COOR, —COR, —SH, —SR, —OH, and halogen atoms,
a modified or unmodified polyorganosiloxane residue,
a polyoxyalkylene group,
wherein R is chosen from saturated or unsaturated, linear, branched or cyclic hydrocarbon-based groups optionally containing at least one atom chosen from nitrogen, oxygen and sulphur atoms, and optionally substituted with at least one group chosen from —OR′, —COOR′, —COR′, —SH, —SR′, —OH, and halogen atoms, and a polymer residue that may be obtained by free-radical polymerization, by polycondensation or by ring opening, R′ being chosen from C₁-C₁₀ alkyl groups.

8. The water-in-oil emulsion of claim 7, wherein the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoagrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate.

9. The water-in-oil emulsion of claim 7, wherein the at least one cyanoacrylate monomer is chosen from C₆-C₁₀ alkyl cyanoacrylates.

10. The water-in-oil emulsion of claim 7, wherein the at least one cyanoacrylate monomer is chosen from octyl cyanoacrylate monomers of formula (V), and mixtures thereof: 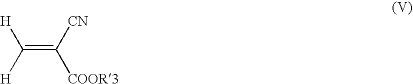
wherein
R′3 is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

11. The water-in-oil emulsion of claim 1, wherein the at least one cyanoacrylate monomer is present in an amount ranging from 0.1% to 80% by weight, relative to the total weight of the emulsion.

12. The water-in-oil emulsion of claim 11, wherein the at least one cyanoacrylate monomer is present in an amount ranging from 0.5% to 50% by weight, relative to the total weight of emulsion.

13. The water-in-oil emulsion of claim 1, wherein the aqueous phase further comprises at least one water-soluble cosmetic active agent.

14. The water-in-oil emulsion of claim 13, where the at least one water-soluble cosmetic active agent is chosen from oxidizing agents, direct dyes, fluorescent dyes, oxidation dyes, polymers and reducing agents.

15. The water-in-oil emulsion of claim 1, wherein the at least one polymerization inhibitor is chosen from sulphur dioxide, nitric oxide, boron trifluoride, hydroquinone and derivatives thereof, benzoquinone and derivatives thereof, catechol and derivatives thereof, anisole and derivatives thereof, pyrogallol and derivatives thereof, p-methoxyphenol, hydroxybutyltoluene, alkyl sulphates, alkyl sulphites, alkyl sulphones, alkyl sulphoxides, alkyl sulphides, mercaptans, 3-sulphonene, organic acids, and mixtures thereof.

16. The water-in-oil emulsion of claim 15, wherein the at least one polymerization inhibitor is acetic acid.

17. The water-in-oil emulsion of claim 1, wherein the at least one surfactant is chosen from non-silicone anionic, cationic, amphoteric and non-ionic surfactants.

18. The water-in-oil emulsion of claim 1, wherein the at least one surfactant is chosen from silicone surfactants.

19. The water-in-oil emulsion of claim 17, wherein the at least one surfactant is present in the emulsion in an amount ranging from 0.1% to 30% by weight, relative to the total weight of the emulsion.

20. A process for treating keratin fibers, comprising:
applying to said keratin fibers a composition comprising a water-in-oil emulsion for the treatment of keratin fibers, said water in oil emulsion comprising an aqueous phase, at least one surfactant, at least one monomer polymerization inhibitor and an oily phase, wherein said oily phase comprises at least one cyanoacrylate monomer capable of polymerizing anionically in the presence of a nucleophilic agent.

21. The process of claim 20, wherein said keratin materials are pretreated with at least one nucleophilic agent.

22. A kit for treating keratin fibers, comprising:
a first composition comprising a water-in-oil emulsion for the treatment of keratin fibers, said water-in-oil emulsion comprising an aqueous phase, at least one surfactant, at least one monomer polymerization inhibitor and an oily phase, wherein said oily phase comprises at least one cyanoacrylate monomer capable of polymerizing anionically in the presence of a nucleophilic agent, and
a second composition comprising at least one nucleophilic agent.

23. A kit for treating keratin fibers, comprising:
a first composition comprising at least one cyanoacrylate monomer capable of polymerizing anionically in the presence of a nucleophilic agent, at least one anionic and/or free-radical polymerization inhibitor and/or an acid, and
a second composition comprising a water-in-oil emulsion comprising an aqueous phase, an oily phase, a surfactant and, optionally, an organic acid.
